(19) United States
(12) Patent Application Publication
(10) Pub. No.: US 2025/0235518 A1
(43) Pub. Date: Jul. 24, 2025

(54) Method for preparing tumor vaccine using magnetic thermal inactivation technology

(71) Applicant: Shaanxi Baici Kangda Medical Technology Co., Ltd., Xi'an (CN)

(72) Inventors: Xiaoli Liu, Xi'an (CN); Haiming Fan, Xi'an (CN); Bin Yan, Xi'an (CN); Yi Lv, Xi'an (CN)

(73) Assignee: Shaanxi Baici Kangda Medical Technology Co., Ltd., Xi'an (CN)

(21) Appl. No.: 19/061,999

(22) Filed: Feb. 24, 2025

Related U.S. Application Data
(63) Continuation of application No. PCT/CN2024/074225, filed on Jan. 26, 2024.

(30) Foreign Application Priority Data
Dec. 20, 2023 (CN) CN202311756049.5

(51) Int. Cl. A61K 39/00; A61K 9/50; A61K 41/10; A61K 45/06; A61P 35/00; C12N 5/09; C12N 13/00; C12Q 1/6806; C12Q 1/6869; C12Q 1/6886

(52) U.S. Cl. CPC A61K 39/0011; A61K 9/5094; A61K 41/10; A61K 45/06; A61P 35/00; C12N 5/0693; C12N 13/00; C12Q 1/6806; C12Q 1/6869; C12Q 1/6886

(57) ABSTRACT

The present invention relates a method for preparing a personalized tumor vaccine using magnetic induction hyperthermia (MIH) inactivation technique, falling within the field of medicine. In this method, a MIH nanoagent is used to generate localized heat within tumor cells upon exposure to an alternating magnetic field, triggering immunogenic cell death and inducing the emergence of neoantigens mutations in tumor cells. Depending on the specific requirements, two strategies can be utilized: one involves preparing a whole tumor cell vaccine containing multiple tumor antigens, while the other focuses on screening specific tumor neoantigens to create a targeted tumor neoantigen vaccine. Mouse model experiments demonstrated that the whole tumor cell vaccine prepared using this method effectively inhibited the growth of homologous tumors, with a tumor-free rate of 100% in the vaccination group. Using MIH for tumor cells inactivation offers several advantages, including the preservation of antigen integrity, enhanced antigen abundance, and an increase in the diversity and quantity of released endogenous adjuvants. All of these factors contribute to the creation of a highly immunogenic personalized tumor vaccine, which holds promise for inhibiting tumor growth, recurrence and metastasis.

### TECHNICAL FIELD

The invention belongs to the field of medical technology, and specifically relates to a method for preparing a tumor vaccine by utilizing magnetic induction hyperthermia inactivation technique.

### BACKGROUND ART

Tumor immunotherapy has become a promising approach in the treatment of various cancers. However, due to the heterogeneity of tumors and individual differences among patients, the clinical response rates of current immunotherapies remain suboptimal, limiting their therapeutic efficacy. Therefore, there is an urgent need to develop more effective tumor immunotherapy strategies.

With an in-depth understanding of tumor biology and immunology, personalized tumor vaccines have shown great potential and have become a key focus in the field of tumor immunotherapy. The primary goal of personalized tumor vaccines is to activate the body's immune system to fight tumors, inhibiting tumor progression and metastasis while preventing recurrence. By delivering specific tumor antigens, these vaccines can specifically activate the immune system to target and destroy tumor cells, while minimizing damage to healthy tissues. Personalized tumor vaccines are created by isolating tumor cells from a patient's resected tumor tissue. These cells are then inactivated ex vivo to prepare tumor cell vaccines, or in some cases, tumor mutations are identified through gene sequencing and big data analysis to design mRNA or peptide-based vaccines, which are subsequently administered for treatment. These vaccines contain a broad spectrum of tumor antigens, including neoantigens, and can bypass T cell tolerance to self-antigens, triggering a strong and durable T cell-mediated immune response. However, current methods of tumor cell inactivation (such as chemical agents, high temperatures, freezing, and radiation) result in vaccines with low immunogenicity, limiting their ability to induce a potent immune response and posing a significant barrier to clinical application. For example, OncoVax®, a tumor cell vaccine developed by Vaccinogen Inc. and approved by the FDA for Phase III clinical trials in colorectal cancer, uses γ-irradiation to inactivate tumor cells and incorporates Bacillus Calmette-Guérin as an adjuvant. Unfortunately, the trial did not yield the expected results, primarily due to the insufficient quantity and integrity of tumor antigens after γ-irradiation. This limitation is primarily due to two factors: (1) Tumor antigens may be lost or altered during the inactivation process, reducing antigen abundance, while immune-suppressive molecules may be exposed, hindering the efficient capture and presentation of antigens; (2) The limited release of endogenous adjuvants fails to significantly enhance immunogenicity. Therefore, the development of innovative inactivation techniques that enhance antigen density while simultaneously releasing abundant endogenous adjuvants is essential for creating highly immunogenic tumor vaccines.

### SUMMARY OF THE INVENTION

In order to overcome the disadvantage of low immunogenicity of tumor antigens in existing tumor vaccines, the purpose of the present invention is to provide a method for preparing tumor vaccines using magnetic induction hyperthermia (MIH) inactivation technique. Using MIH for tumor cells inactivation has the advantages of preserving antigen integrity, enhancing antigen abundance, and increasing the diversity and quantity of released endogenous adjuvants, while simultaneously reducing the levels of immune-suppressive molecules. According to specific treatment needs, the MIH inactivation technique described in the present invention can be used to prepare two types of vaccines: whole tumor cell vaccines and tumor neoantigen vaccines. Whole tumor cell vaccines contain a diverse array of tumor antigens, enabling the stimulation of broad immune responses, while tumor neoantigen vaccines target specific, individualized neoantigens and provide precise and tailored immune responses. By employing this approach, the present invention not only improves the efficacy of tumor vaccines, but also provides new possibilities for personalized tumor treatment.

To achieve the above object, the technical solution adopted by the present invention is:

First, MIH nanoagents are used to generate localized heat within tumor cells upon exposure to an alternating magnetic field, triggering immunogenic cell death and inducing the emergence of neoantigens mutations in tumor cells. Following this process, two distinct strategies can be implemented based on specific therapeutic needs: one approach involves the preparation of highly immunogenic whole tumor cell vaccines containing a broad spectrum of tumor antigens, while the other focuses on screening specific tumor neoantigens to develop precisely targeted tumor neoantigen vaccines.

In one embodiment, tumor cells or ex vivo tumor tissue of a patient undergo MIH inactivation treatment, which includes the following steps:
Step 1: Tumor cells internalize MIH nanoagents, or disperse ex vivo tumor tissue into single cell suspension, sort and expand tumor cells to internalize MIH nanoagents.
Step 2: Apply an alternating magnetic field to the tumor cells that have internalized the MIH nanoagents, so that the MIH nanoagents generate localized heat in the cells, triggering immunogenic cell death and inducing the emergence of neoantigens mutations in tumor cells.
Step 3: The tumor cells and their lysates resulting from MIH inactivation are collected, separated and purified to obtain highly immunogenic substances. Alternatively, specific tumor neoantigens are screened and selected. These immunogenic substances or neoantigens are then used to prepare personalized tumor vaccines.

In one embodiment, after step 2, the tumor cells and their lysates resulting from MIH inactivation are collected, separated and purified to obtain highly immunogenic substances, which are then used to prepare whole tumor cell vaccines containing a diverse array of tumor antigens.

In one embodiment, after step 2, specific tumor neoantigens are screened, and the highly immunogenic tumor neoantigens produced after MIH inactivation are collected, separated and purified, and to prepare targeted tumor neoantigen vaccines.

In one embodiment, a method for screening tumor neoantigens for preparing a tumor neoantigen vaccine comprises the following steps:
Step 1: Extract DNA and RNA from tumor cells subjected to MIH inactivation, perform exome sequencing and transcriptome sequencing, and conduct a preliminarily screening for tumor neoantigens.
Step 2: Conduct an immunogenicity test on the preliminarily screened tumor neoantigens to identify those with high immunogenicity.

In one embodiment, the MIH inactivation technique is performed at a concentration of 50-1000 μg/mL, with an alternating magnetic field strength of 10-1000 Oe, a frequency of 50 kHz-1 MHz, an action duration of 10-60 minutes, and a temperature range of 39-49° C. MIH exerts a dual synergistic effect of thermal and oxidative stress within lysosomes, efficiently inducing immunogenic cell death and generating neoantigens epitopes.

In one embodiment, the MIH inactivation technique is used to prepare a tumor neoantigen vaccine, with the concentration of the tumor neoantigen is 20-2000 μg/μL.

In one embodiment, the tumor cell is selected from at least one of the following: liver cancer cells, lung cancer cells, breast cancer cells, colon cancer cells, pancreatic cancer cells, prostate cancer cells, gastric cancer cells, kidney cancer cells, or melanoma cells.

In one embodiment, personalized tumor vaccines are used alone or in combination with clinically used anti-tumor modalities (e.g., surgery, chemotherapy, radiotherapy, photothermal therapy, photodynamic therapy, etc.) to inhibit tumor growth, recurrence, and metastasis.

In one embodiment, the MIH nanoagent is selected from Fe, FeCo, Fe₂C, FePt, MFe₂O₄ or metal-doped iron-based magnetic nanoparticles, wherein M is Fe, Mn, Co, Ni or Zn. The nanomaterials have a particle size ranging from 3 to 500 nanometers and exhibit at least one of a spherical, cubic, disk, or ring morphology.

In one embodiment, the surface of the MIH nanoagent is coated with a hydrophilic or amphiphilic polymer to facilitate dispersion in an aqueous solution. The polymer is selected from at least one of 3,4-dihydroxyhydrocinnamic acid, polyethylene glycol, or dopamine.

MIH is a clinical tumor treatment modality that uses MIH nanoagents as a heat source. These nanoagents generate heat when exposed to an alternating magnetic field, thereby destroying tumors. Compared to other clinical thermal therapies, such as microwave or radiofrequency treatments, MIH offers the advantage of utilizing nano-sized thermal sources that can enter tumor cells, achieving an intracellular thermal therapy, which is much more efficient than conventional thermal treatments. Tumor cells treated with MIH lose their proliferative ability and show high immunogenicity, making MIH an ideal technique for the preparation of whole tumor cell vaccines. In addition, tumor neoantigens generated through MIH can be further screened for the preparation of tumor neoantigen vaccines. These vaccines can enhance the duration of specific T cell responses and provide immune memory after treatment, contributing to the long-term prevention of tumor recurrence.

Compared with the prior art, the MIH inactivation technique of the present invention has significant beneficial effects:
1. Enhance the immunogenicity of tumor cells: MIH exerts a dual synergistic effect of thermal and oxidative stress within lysosomes, effectively inducing immunogenic cell death. This process leads to the release of various endogenous adjuvants, such as CRT, HMGB1, ATP, significantly enhancing immunogenicity.
2. Generate tumor neoantigens: MIH induces multiple forms of tumor cell death, including necroptosis, pyroptosis, and ferroptosis, each of which facilitates the release of tumor antigens and the generation of neoantigens.
3. Retain the integrity of antigenic epitopes: Based on the principle that the thermal energy generated by a single MIH nanoagent matches the weak hydrogen bond energy of higher-order protein structures, MIH disrupts these structures without compromising the integrity of antigenic epitopes.
4. MIH downregulates immune-suppressive molecules, thereby promoting tumor antigen uptake and presentation
5. Wide applicability: The present invention is applicable to the preparation of various types of solid tumor vaccines, showing a high degree of versatility.

The present invention provides a new personalized tumor vaccine strategy, which is of great significance to clinical tumor immunotherapy and indicates its broad application prospects in the field of cancer treatment.

### BRIEF DESCRIPTION OF THE DRAWINGS

FIG. 1 is a schematic diagram of a method for preparing tumor vaccines using MIH inactivation technique.

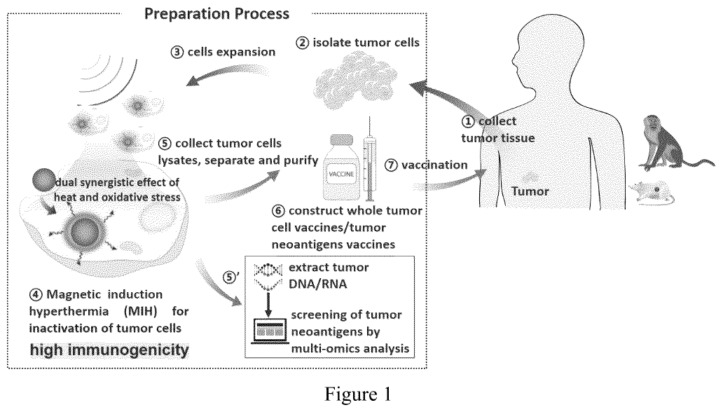

FIG. 2 shows the immunofluorescence results of calreticulin externalization in Hepa1-6 cells after MIH treatment.

FIG. 3 shows the content of HMGB1 in the supernatant of Hepa1-6 cell culture medium after MIH treatment.

FIG. 4 shows the ATP content in the supernatant of Hepa1-6 cell culture medium after MIH treatment.

FIG. 5 shows the cell activity of Hepa1-6 cells after MIH treatment.

FIG. 6 shows the preventive effect of whole tumor cell vaccines on homologous tumor cells.

### DETAILED DESCRIPTION

The embodiments of the present invention are described in detail below with reference to the accompanying drawings and examples.

### Example 1

### Induction of Immunogenic Death of Tumor Cells by MIH

### 1. Cell Culture and Treatment

Hepa 1-6 cells were obtained from ATCC. Hepa 1-6 cells were cultured in RPMI-1640 medium supplemented with 10% fetal bovine serum, 100 μg/mL streptomycin, and 100 μg/mL penicillin. Hepa 1-6 cell cultures were maintained at 37° C. and 5% CO₂.

### 2. MIH Treatment

When the tumor cells grow to 80%, add 50-1000 μg/mL magnetic iron oxide nanoparticles are incubated with cells for 6-12 hours, and the incubated cells are placed in an alternating magnetic field (10-1000 Oe, 50 kHz-1 MHZ) for 10-60 minutes. The treated cells are cultured for 0-24 hours for later use.

### 3. MIH Induces Calreticulin Exposure

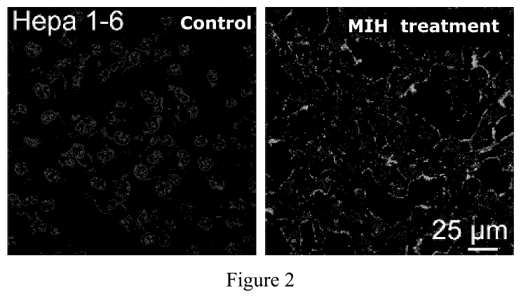

When tumor cells undergo immunogenic cell death, calreticulin will be exposed on the cell membrane, acting as an “eat-me” signal that facilitates the phagocytosis of dying tumor cells by dendritic cells or their precursors. This process not only provides rich antigenic substances but also promotes the maturation and functional activation of dendritic cells. In this experiment, cells treated with MIH were initially blocked with 5% BSA for 15 minutes. After discarding the blocking solution, and the cells were washed three times with PBS. The cells were then incubated with recombinant Alexa Fluor® 488-conjugated Anti-Calreticulin antibody (Abcam) at 4° C. for 30 minutes in the dark. The cell nucleus was stained with DAPI. Imaging was performed using a confocal microscope (NIS-Elements, Ti2). As shown in FIG. 2, the experimental results demonstrate that MIH effectively induces calreticulin exposure from the intracellular compartment to the cell membrane, promoting its “eat-me” signaling function for immune system activation.

### 4. MIH Induces HMGB1 and ATP Release

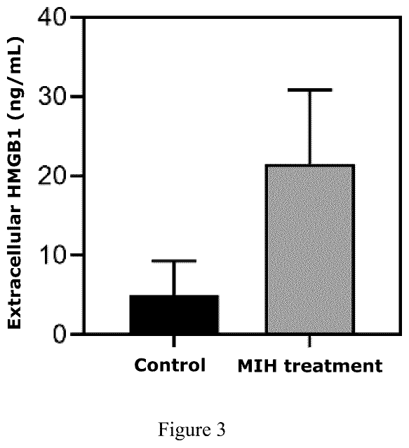

When tumor cells undergo immunogenic cell death, HMGB1 is released into the extracellular space, where it activates specific signaling pathways and enhances immune responses. In this experiment, the supernatant of the cell culture medium was collected following MIH treatment and centrifuged at 4° C. at 300×g for 3 minutes to remove residual cells or cell debris. The content of extracellular HMGB1 was quantified using the Mouse HMGB1/HMG-1 ELISA Kit (Novus Biologicals). As shown in FIG. 3, experimental results demonstrate that MIH treatment led to a 4.32-fold increase in extracellular HMGB1 levels.

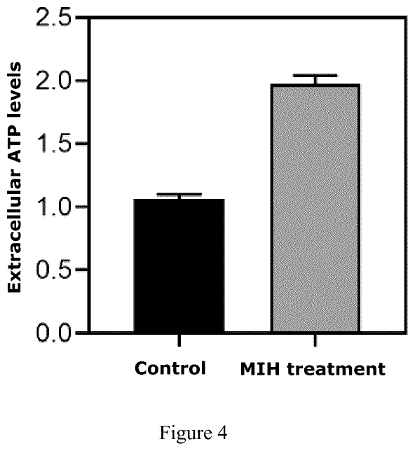

During apoptosis, tumor cells release intracellular ATP into the extracellular space, acting as a “find-me” signal to attract phagocytes. This extracellular ATP promotes the phagocytosis of dying cells and stimulates specific anti-tumor immune responses. The level of extracellular ATP in the culture medium supernatant was measured by an ATP detection kit (Solebo). As shown in FIG. 4, the results demonstrate that after MIH treatment, the extracellular ATP content increased by 1.97-fold, further enhancing immune activation.

### Example 2

### Inhibition of Tumor Cell Activity by MIH

Hepa 1-6 cells in a healthy growth state during the logarithmic phase were treated with varying concentration of a MIH nanoagent in the cell culture medium. The cells were then subjected to MIH treatment under an alternating magnetic field to evaluate the cytotoxic effects of MIH. Specifically:

Hepa 1-6 cells in the logarithmic phase were harvested and digested into a single-cell suspension using trypsin containing 0.25% EDTA. After counting the cells with a hemocytometer, 5×10⁵ cells were seeded into 35 mm cell culture dishes and inoculated at 37° C. with 5% CO₂ for 24 hours. Once the cells adhered to the dish surface, the culture medium was removed, and MIH nanoagent of varying concentrations were added. The cells were then cultured further to allow internalization of the nanoagents. After MIH treatment in an alternating magnetic field, the cells were digested again into a single-cell suspension with trypsin containing 0.25% EDTA, and cell counts were obtained using a hemocytometer. The resulting cell suspension was plated into a 96-well plate, with 1×10⁴ cells per well, and incubated at 37° C. with 5% CO₂ for 24 hours. CCK-8 reagent (100 μL) was then added to each well, and cell viability was assessed by measuring the OD value using a microplate reader with dual-wavelength analysis (detection wavelength: 450-490 nm; reference wavelength: 600-650 nm).

Cell viability was calculated using the following formula:

$$Cell viability = ( {OD}_{experimental} - {OD}_{control} ) / ( {OD}_{control -} {OD}_{blank} ) \times 100 \% .$$

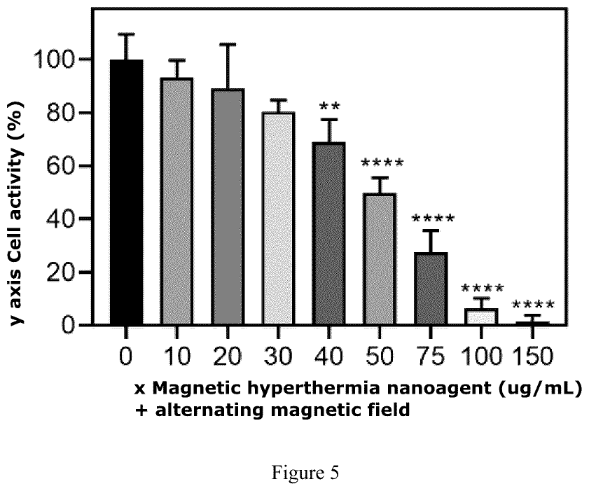

As shown in FIG. 5, when the concentration of the MIH nanoagent was 100 μg/mL, cell viability was reduced to below 10%. As the concentration of MIH nanoagents increased, MIH was shown to effectively inactivate tumor cells.

### Example 3

### Screening of Tumor Neoantigens

\textcircled{1} Extraction of total DNA from tumor cells: The tumor cells were first washed with TBS, then centrifuged at 4000×g for 5 minutes. The supernatant was discarded. Next, 10 times the volume of lysis buffer was added, and the sample was incubated in a water bath at 50-55° C. for 1-2 hours. To the lysate, an equal volume of saturated phenol was added, and the solution was mixed thoroughly, followed by a 3-minute standing period. The mixture was then centrifuged at 5000×g for 10 minutes, and the upper aqueous phase was transferred to a new 1.5 mL EP tube. An equal volume of phenol/chloroform was added, mixed gently, and centrifuged at 5000×g for 10 minutes. The upper aqueous phase was transferred again into a new EP tube. An equal volume of chloroform was added, mixed gently, and centrifuged again at 5000×g for 10 minutes. The upper aqueous phase was collected in a separate EP tube. To this, 1/10 volume of 3 M sodium acetate (pH=5.2) and 2.5 times the volume of anhydrous ethanol were added. The solution was gently inverted to mix. Upon the appearance of precipitates, the sample was centrifuged at 5000×g for 5 minutes, and the supernatant was discarded. The precipitate was washed with 75% ethanol, centrifuged at 5000×g for 3 minutes, and the ethanol was discarded. The ethanol was allowed to evaporate at room temperature, and 50 μL TE buffer was added to dissolve the DNA. The resulting solution contained tumor DNA.
\textcircled{2} Extraction of tumor cell RNA: Total RNA was extracted using the TRIzol method. Adherent cells were washed with PBS, and 1 mL of TRIzol was added to the culture dish. The cells were gently pipetted until fully lysed, and the lysate was transferred to a 2 mL EP tube. The mixture was inverted and incubated at room temperature for 5 minutes. Then, 200 μL of chloroform was added to the tube, mixed by inversion for 30 seconds, and incubated at room temperature for 5 minutes. The sample was centrifuged at 4° C., 12,000× g for 15 minutes. The upper aqueous phase was transferred to a new EP tube, to which an equal volume of isopropanol was added, mixed, and incubated at room temperature for 10 minutes. The solution was centrifuged at 4° C., 12000×g for 10 minutes, and the supernatant was discarded. Next, 1 mL of cold 75% ethanol (prepared with DEPC water) was added to wash the RNA pellet, and the precipitate was resuspended. The sample was centrifuged again at 4° C., 7500× g for 5 minutes, and the precipitate was retained. This washing step was repeated twice. After opening the EP tube in a sterile, enzyme-free environment, the RNA was dried at room temperature until the ethanol evaporated. Finally, 20 μL of DEPC water was added, and the solution was gently pipetted to dissolve the RNA. The resulting solution contained total cellular RNA.
\textcircled{3} Transcriptome and Exome Sequencing: Transcriptome sequencing was performed on the extracted RNA, while exome sequencing was performed on the extracted DNA. The sequencing data were analyzed to identify tumor-specific mutations. Dominant mutant peptides were then selected based on HLA affinity. Finally, in vitro T cell reactivity assays were conducted to assess the immunogenicity of the selected peptides.

### Example 4

### Tumor Vaccine Preparation Process

An appropriate quantity of tumor cells or digested and dispersed tumor tissues is first cultured in vitro. MIH nanoagents are added to the culture, ensuring complete internalization by the tumor cells. After the cells have fully internalized the MIH nanoagents, an alternating magnetic field is applied to induce tumor immunogenic cell death. This MIH inactivation process is carefully performed to achieve the desired tumor cell destruction. Next, highly immunogenic tumor cells and their lysates are separated and purified. Optimize this preparation process and establish a standardized quality control system for whole tumor cell vaccines, providing a foundation for large-scale production and clinical translation.

Alternatively, DNA and RNA are extracted from the tumor cells after MIH inactivation. Multi-omics analysis is then used to screen tumor neoantigens produced following MIH inactivation. The immunogenicity these neoantigens is tested to determine one or more highly immunogenic tumor neoantigens. For the tumor neoantigen vaccine, the final preparation contains 20-2000 μg/μL of tumor neoantigens. The prepared tumor vaccine is sterilized, tested for pathogens, and packaged for storage at 4° C.

### Example 5

### Whole Tumor Cell Vaccines on Homologous Tumors

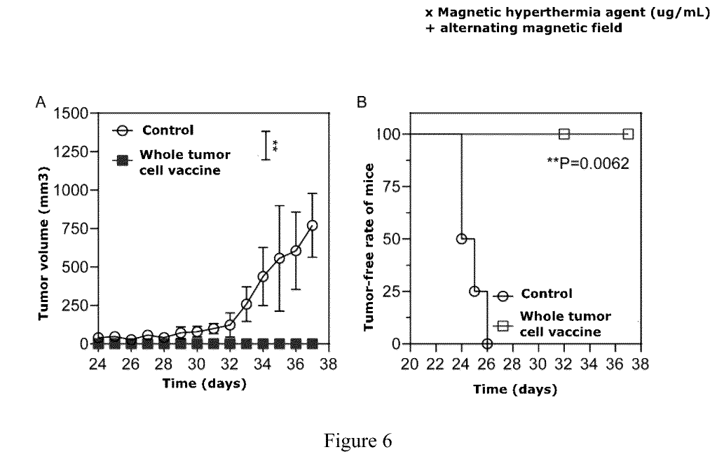

The tumor vaccine experiment used 6-8 week-old male C57BL/6 mice (purchased from Beijing Weitong Lihua Experimental Animal Technology Co., Ltd.) to evaluate the resistance of the whole tumor cell vaccine against homologous tumors. Twelve mice were randomly divided into two groups (the control group and the vaccination group). On days 0, 7, and 14 of the experiment, each mouse in the vaccination group was subcutaneously injected with whole tumor cell vaccine (left back), while the control group received an injection of normal saline. On day 21, each mouse was injected with 1×10⁶ Hepa1-6 cells (right back), and the growth of tumors was subsequently monitored, as shown in FIG. 6A. The results revealed that in the control group, after the injection of normal homologous tumor cells, the tumors grew, and the tumor-free rate was 0%. In contrast, no tumor growth was observed in the vaccination group for 38 consecutive days, resulting in a 100% tumor-free rate (FIG. 6B). These findings demonstrate that the whole tumor cell vaccine, prepared using MIH inactivation, exhibits excellent tumor prevention efficacy and significantly inhibits the growth of homologous tumors.

It should be pointed out that a person skilled in the art can make several modifications and improvements without departing from the inventive concept of the present invention, and these modifications and improvements all fall within the protection scope of the present invention.

1. A method for preparing a personalized tumor vaccine using MIH inactivation technique, characterized in that the personalized tumor vaccine is a whole tumor cell vaccine or a tumor neoantigen vaccine, and the preparation process is as follows:
Step 1, cause tumor cells to internalize MIH nanoagents, by dispersing ex vivo tumor tissue into single cell suspension, sorting and expanding tumor cells, and then allowing the cells to internalize magnetic hyperthermia agents;
Step 2, apply an alternating magnetic field to cause the magnetic hyperthermia agent to generate magnetic heat in the cells, triggering immunogenic death of tumor cells and inducing new antigen mutations in tumor cells;
Step 3, The tumor cells and their lysates resulting from MIH inactivation are collected, separated and purified to obtain highly immunogenic substances. Alternatively, specific tumor neoantigens are screened and selected. These immunogenic substances or neoantigens are then used to prepare personalized tumor vaccines;
Step 4: Perform one of the following steps:
Step 4a, Collect, separate and purify tumor cells and their lysates to obtain highly immunogenic substances, which are then used to prepare whole tumor cell vaccines containing a diverse array of tumor antigens;
Step 4b: Screen specific tumor neoantigens, collect, separate and purify them, which are then used to prepare targeted tumor neoantigen vaccines.

2. The method for preparing tumor vaccines using MIH inactivation technique according to claim 1, characterized in that the tumor cells are selected from at least one of the following: liver cancer cells, lung cancer cells, breast cancer cells, colon cancer cells, pancreatic cancer cells, prostate cancer cells, gastric cancer cells, kidney cancer cells, or melanoma cells.

3. The method for preparing tumor vaccines using MIH inactivation technique according to claim 1, characterized in that the MIH nanoagent is selected from Fe, FeCo, Fe₂C, FePt, MFe₂O₄ or metal-doped iron-based magnetic nanoparticles, wherein M is Fe, Mn, Co, Ni or Zn. The nanomaterials have a particle size ranging from 3 to 500 nanometers and exhibit at least one of a spherical, cubic, disk, or ring morphology.

4. The method for preparing tumor vaccines using MIH inactivation technique according to claim 3, characterized in that the surface of the MIH nanoagent is coated with a hydrophilic or amphiphilic polymer to facilitate dispersion in an aqueous solution.

5. The method for preparing tumor vaccines using MIH inactivation technique according to claim 4, characterized in that the polymer is selected from at least one of 3,4-dihydroxyhydrocinnamic acid, polyethylene glycol, or dopamine.

6. The method for preparing a tumor vaccine using MIH inactivation technique according to claim 1, characterized in that the concentration range of the MIH inactivation technique is performed at a concentration of 50-1000 μg/mL, with an alternating magnetic field strength of 10-1000 Oe, a frequency of 50 kHz-1 MHz, an action duration of 10-60 minutes, and a temperature range of 39-49° C. MIH exerts a dual synergistic effect of thermal and oxidative stress within lysosomes, efficiently inducing immunogenic cell death and generating neoantigens epitopes.

7. The method for preparing tumor vaccines using MIH inactivation technique according to claim 1, characterized in that the method for screening specific tumor neoantigens is: extracting DNA and RNA from tumor cells subjected to MIH inactivation, performing exome sequencing and transcriptome sequencing, and conducting a preliminarily screening for tumor neoantigens.

8. The method for preparing tumor vaccines using MIH inactivation technique according to claim 8, characterized in that the immunogenicity of the tumor neoantigens preliminarily screened out is tested to screen out tumor neoantigens with high immunogenicity.

9. The method for preparing a tumor vaccine using MIH inactivation technique according to claim 1, characterized in that the concentration of the tumor neoantigen in the tumor neoantigen vaccine is 20-2000 μg/μL.

10. The method for preparing a tumor vaccine using MIH inactivation technique according to claim 1, characterized in that the personalized tumor vaccines are used alone or in combination with clinically used anti-tumor modalities (e.g., surgery, chemotherapy, radiotherapy, photothermal therapy, photodynamic therapy, etc.) to inhibit tumor growth, recurrence, and metastasis.
